(19) United States
(12) Patent Application Publication
(10) Pub. No.: US 2023/0290496 A1
(43) Pub. Date: Sep. 14, 2023

(54) TREATMENT AID APPARATUS

(71) Applicant: TERUMO KABUSHIKI KAISHA, Tokyo (JP)

(72) Inventors: Yuichi TADA, Santa Clara, CA (US); Yoichiro KUWANO, Machida Tokyo (JP)

(21) Appl. No.: 18/177,648

(22) Filed: Mar. 2, 2023

(30) Foreign Application Priority Data
Mar. 9, 2022 (JP) 2022-036347

(51) Int. Cl. G16H 40/63; A61B 17/3207

(52) U.S. Cl. CPC G16H 40/63; A61B 17/320758; G06F 3/0482

(57) ABSTRACT

A medical system for removing an object in a body cavity, includes a display, and a controller connectable to a drive device configured to control a medical device operated by an operator to remove the object. The controller is configured to acquire device information indicating features of different types of first devices connectable to the medical device, acquire target information indicating characteristics of a target object to be removed, rank the first devices for removing the target object using the device information and the target information, and display on the display a list of the first devices according to the rank thereof.

### CROSS-REFERENCE TO RELATED APPLICATION(S)

This application is based on and claims the benefit of priority from Japanese patent application No. 2022-036347, filed Mar. 9, 2022, the entire contents of which are incorporated herein by reference.

### BACKGROUND

### Technical Field

Embodiments described herein relate generally to a medical system and a treatment aid apparatus for aiding an operator of a medical device to remove an object in a cavity of a biological body.

### Related Art

In recent years, atherectomy devices have been used for cutting a lesion such as plaque and thrombus in a cavity of a biological body such as a blood vessel. Such atherectomy devices have a cutting unit configured to cut a lesion inside a blood vessel.

### SUMMARY

Selection of an atherectomy device or a treatment method suitable for each patient is based on the personal experience of a physician who is an operator of the device. However, symptoms vary from patient to patient, and it is not easy to select an optimal device or an optimal treatment method.

Embodiments of the invention provide a treatment aid apparatus capable of automatically determining a device and a treatment method appropriately individualized for a lesion of each patient and reducing burden on an operator.

In one embodiment, a medical system for removing an object in a body cavity, includes a display, and a controller connectable to a drive device configured to control a medical device operated by an operator to remove the object. The controller is configured to acquire device information indicating features of different types of first devices connectable to the medical device, acquire target information indicating characteristics of a target object to be removed, rank the first devices for removing the target object using the device information and the target information, and display on the display a list of the first devices according to the rank thereof.

In another embodiment, a treatment aid apparatus for aiding an operator of a medical device to remove an object in a body cavity, includes a display, and a processor configured to: acquire device information indicating features of different types of first devices connectable to the medical device, acquire target information indicating characteristics of a target object to be removed, rank the first devices for removing the target object using the device information and the target information, and control the display to display a list of the first devices according to the rank thereof.

In yet another embodiment, a treatment aid apparatus for aiding an operator of a medical device to remove an object in a body cavity, includes a display, and a processor configured to: acquire treatment method information indicating features of different types of methods for removing an object in a body cavity, acquire target information indicating characteristics of a target object to be removed, rank the treatment methods for removing the target object using the treatment method information and the target information, and control the display to display a list of the methods according to the rank thereof.

The treatment aid apparatus having the above configuration automatically determines types of devices or treatment methods appropriately individualized for a lesion of each patient and causes the display to show the information, so that it is possible to reduce burden on the operator.

### BRIEF DESCRIPTION OF DRAWINGS

FIG. 1 is an overall front view of a medical system including a treatment aid apparatus according to a first embodiment.

FIG. 2 is an enlarged cross-sectional view of a distal end portion of an atherectomy device of the medical system.

FIG. 3 is a hardware block diagram of the medical system.

FIG. 4 is a flowchart showing an operation performed by a controller of the medical system.

FIG. 5 illustrates a screen showing different types of recommended atherectomy devices in a selectable manner.

FIG. 6 illustrates a screen showing different types of recommended access devices in a selectable manner.

FIG. 7 illustrates a screen showing different types of recommended treatment devices in a selectable manner.

FIG. 8 illustrates a screen showing different types of recommended treatment methods in a selectable manner.

FIG. 9 is a cross-sectional view showing a cutting unit positioned near a lesion in a body cavity.

FIG. 10 is a functional block diagram of a controller of a treatment aid apparatus according to a second embodiment.

FIG. 11 is a flowchart showing an operation of the controller of the treatment aid apparatus in the second embodiment.

### DETAILED DESCRIPTION

Hereinafter, embodiments of the invention will be described with reference to the accompanying drawings. Note that the size and ratio of each member in the drawings may be exaggerated for illustration and may be different from the actual size and ratio.

### First Embodiment

A treatment aid apparatus 110 according to a first embodiment aids an operator in performing a treatment to cut an object such as a lesion within a blood vessel by determining an appropriate atherectomy device 10 used for the treatment from different types of atherectomy devices 10. In cases of acute limb ischemia and deep thrombophlebitis, an atherectomy device 10 is inserted into a blood vessel and used for destroying and removing a lesion such as thrombus, plaque, atheroma, and calcified lesion. Herein, a side of the device to be inserted into a blood vessel is referred to as “distal side” and a side to be operated is referred to as “proximal side.” Note that a lesion to be destroyed and removed is not necessarily limited to thrombus, plaque, atheroma, and calcified lesion and may be any object that may exist in a cavity of a biological body.

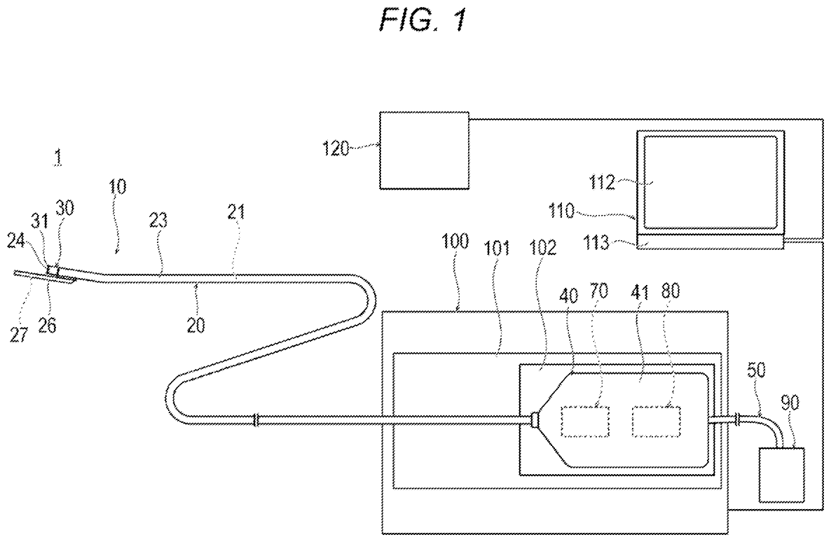

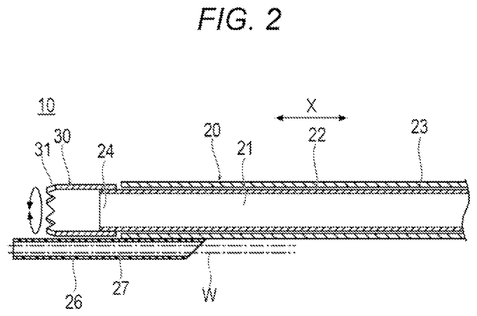

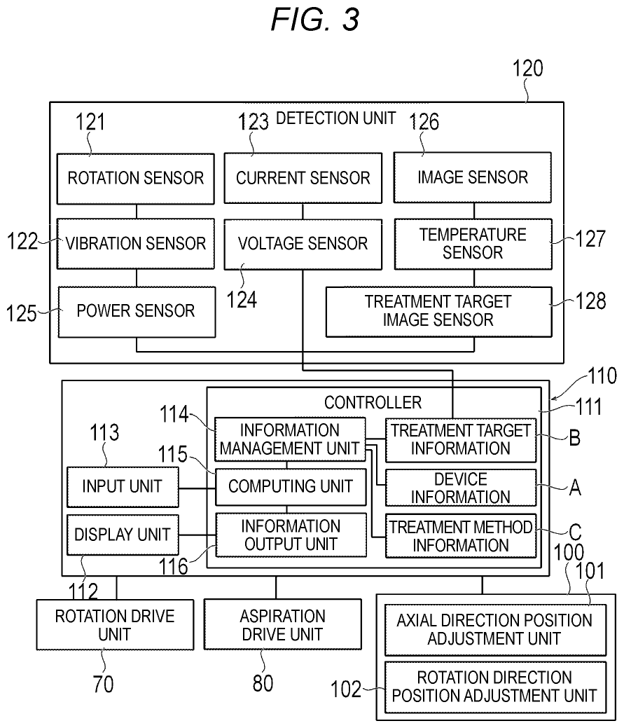

As shown in FIGS. 1 to 3, the treatment aid apparatus 110 is included in a medical system 1. The medical system 1 includes the atherectomy devices 10 each having a distal end portion to which a cutting unit 30 is attached for destroying a lesion, a drive device 100 configured to adjust the position of each atherectomy device 10, a detection unit 120 configured to detect information associated with the atherectomy devices 10, and the treatment aid apparatus 110.

Each atherectomy device 10 includes an elongated shaft portion 20 including the cutting unit 30 disposed in a distal end portion thereof, an extraction lumen 21 that allows an object that has been cut by the cutting unit 30 to pass toward the proximal side, shaft portion 20 and an operation unit 40 to which a proximal end portion of the shaft portion 20 is coupled. Each atherectomy device 10 also includes a rotation drive unit 70 configured to rotate a drive shaft 22 disposed in the shaft portion 20, an aspiration drive unit 80 communicating with the extraction lumen 21 disposed in the shaft portion 20, a discharge channel 50 through which waste fluid aspirated by the aspiration drive unit 80 is discharged, and a drainage bag 90 communicating with the discharge channel 50 and configured to receive the waste fluid through the discharge channel 50.

The shaft portion 20 includes the drive shaft 22 which is rotatably driven by the rotation drive unit 70, an outer tube 23 that rotatably houses the drive shaft 22, and a distal tube 26 fixed to a side surface of a distal end portion of the outer tube 23.

The drive shaft 22 is coupled to the cutting unit 30 and transmits torque to the cutting unit 30. The drive shaft 22 is flexible and has a property of transmitting torque applied from the proximal side to the distal side. The drive shaft 22 includes the extraction lumen 21 for moving a cut object toward the proximal side. The drive shaft 22 penetrates the outer tube 23 and has a distal end portion to which the cutting unit 30 is fixed. The drive shaft 22 has a proximal end portion coupled to the rotation drive unit 70. At the distal end of the drive shaft 22, a distal opening 24 of the extraction lumen 21 is formed. The distal opening 24 is an inlet through which a piece of an object that has been cut and is to be aspirated, enters. The proximal end portion of the drive shaft 22 is coupled to the aspiration drive unit 80 configured to apply a suction force to the extraction lumen 21. Note that the extraction lumen 21 is not necessarily disposed inside the drive shaft 22 and may be disposed between the outer tube 23 and the drive shaft 22 or in another tube disposed inside the drive shaft 22.

The cutting unit 30 is a cutter configured to cut and reduce the size of an object such as thrombus, plaque, and calcified lesion. Therefore, the term “cut” represents the action of applying a force to an object by the cutting unit 30 to reduce the size of the object. A method for applying a force during the cutting and the shape or figure of the cut object are not limited. The cutting unit 30 has a strength capable of cutting the object. The cutting unit 30 is fixed to the distal end portion of the drive shaft 22. The cutting unit 30 is a cylinder protruding toward the distal side further than the drive shaft 22. The cutting unit 30 may have a hollow structure communicating with the extraction lumen 21. The cutting unit 30 has a sharp blade at the distal end thereof. Note that the blade is not particularly limited in shape. The cutting unit 30 may have a large number of fine abrasive grains instead of the blade.

The rotation drive unit 70 is disposed inside the operation unit 40 and configured to rotate the drive shaft 22. The rotation drive unit 70 is, for example, a motor. The rotation drive unit 70 is not particularly limited in rotation speed and may rotate at, for example, 5,000 to 200,000 rpm.

The aspiration drive unit 80 is disposed inside the operation unit 40. The aspiration drive unit 80 is, for example, a pump communicating with a proximal end portion of the extraction lumen 21 of the drive shaft 22 and is configured to apply a suction force (i.e., negative pressure) to the extraction lumen 21. The aspiration drive unit 80 moves waste fluid aspirated through the extraction lumen 21 in the downstream direction and discharges the waste fluid to the drainage bag 90. The aspiration drive unit 80 is, for example, a peristaltic pump or a diaphragm pump. A peristaltic pump partially squeezes a tube with a plurality of rotating rollers and shifts the squeezed position to move waste fluid inside the tube.

The outer tube 23 is a flexible tube that rotatably houses the drive shaft 22. The outer tube 23 has a proximal end portion connected to the operation unit 40. The distal end portion of the outer tube 23 may include a curved portion that bends at a predetermined angle. With this configuration, by rotating the outer tube 23, it is possible to change the orientation of the cutting unit 30 that is positioned on the distal side of the curved portion of the outer tube 23 so that the cutting unit 30 comes into contact with an object easily.

The distal tube 26 is a flexible tube fixed to the outer periphery of the distal end portion of the outer tube 23. The distal tube 26 includes a guide wire lumen 27 that allows a guide wire W to insert therethrough. The rotation of the outer tube 23 changes the rotation direction of the distal tube 26 relative to the outer tube 23.

The discharge channel 50 is disposed between the aspiration drive unit 80 and the drainage bag 90 and through which the waste fluid is discharged from the aspiration drive unit 80 to the drainage bag 90. The discharge channel 50 is preferably transparent or translucent to enable an operator to observe a flow of the waste liquid inside the discharge channel 50.

The drive device 100 includes an axial direction position adjustment unit 101 configured to move the shaft portion 20 along the axial direction X and a rotation direction position adjustment unit 102 configured to rotate the shaft portion 20 about the axial direction X.

The axial direction position adjustment unit 101 includes a linear motion mechanism configured to hold the operation unit 40 and/or the shaft portion 20 of each atherectomy device 10 and couple those members, thereby moving the operation unit 40 and/or the shaft portion 20 along the axial direction X. The linear motion mechanism may include a roller configured to rotate so as to move the shaft portion 20 in contact with the outer periphery of the linear motion mechanism. The operation of the linear motion mechanism is controlled by a controller 111 of the treatment aid apparatus 110. Accordingly, the axial direction position adjustment unit 101 moves the shaft portion 20 and the cutting unit 30 along the axial direction X according to the instructions or control signals issued by the controller 111.

The rotation direction position adjustment unit 102 includes a rotation drive mechanism configured to hold the operation unit 40 and/or the shaft portion 20 of each atherectomy device 10 and couple those members, thereby rotating the operation unit 40 and/or the shaft portion 20 about the axis direction X. The operation of the rotation drive mechanism is controlled by the controller 111. Accordingly, the rotation direction position adjustment unit 102 rotates the shaft portion 20 about the axis direction X according to the instructions or control signals issued by the controller 111. Note that the drive device 100 including the axial direction position adjustment unit 101 and the rotation direction position adjustment unit 102 may be, for example, a robot arm movable in three dimensions.

The detection unit 120 that detects information associated with the atherectomy devices 10 includes a rotation sensor 121, a vibration sensor 122, a current sensor 123, a voltage sensor 124, a temperature sensor 127, a power sensor 125, an image sensor 126, and a treatment target image sensor 128.

The number of rotations of the cutting unit 30 is detected by the rotation sensor 121 disposed in the detection unit 120. The rotation sensor 121 may be disposed in each atherectomy device 10 or the drive device 100. Note that the rotation sensor 121 does not necessarily detect the rotation of the cutting unit 30 directly and may detect the number of rotations of the drive shaft 22 or the rotation drive unit 70 and use the detection result to determine the number of rotations of the cutting unit 30. The rotation sensor 121 is not particularly limited and may be, for example, an optical tachometer or a magnetic tachometer.

Sounds and vibrations produced by the cutting unit 30 and the rotation drive unit 70 are detected by the vibration sensor 122 disposed in the detection unit 120 or near the cutting unit 30, the rotation drive unit 70 of each atherectomy device 10, or the drive device 100. The vibration sensor 122 is not particularly limited and may be, for example, a microphone that detects sounds or an accelerometer that detects vibrations.

A value of current flowing to the rotation drive unit 70 is detected by the current sensor 123 disposed in the detection unit 120, each atherectomy device 10, or the drive device 100. The current sensor 123 is, for example, an ammeter.

An amount of energy supplied to the rotation drive unit 70 is detected by the current sensor 123 and the voltage sensor 124 disposed in the detection unit 120, each atherectomy device 10, or the drive device 100. The voltage sensor 124 is, for example, a voltmeter. In addition, it is possible to compute torque from the amount of energy and the number of rotations.

A temperature around the rotation drive unit 70 is detected by the temperature sensor 127 disposed in the detection unit or near the rotation drive unit 70 of each atherectomy device 10 or the drive device 100. The temperature sensor 127 is, for example, a thermocouple or an infrared temperature sensor.

A reaction force received by each atherectomy device 10 and a value of frictional resistance between each atherectomy device 10 and the drive device 100 are detected by, for example, the power sensor 125 disposed in the detection unit 120, each atherectomy device 10, or the drive device 100. The power sensor 125 is, for example, a gauge type sensor or a piezoelectric sensor.

A value of frictional resistance between each atherectomy device 10 and the drive device 100 is detected by a gauge type sensor or a piezoelectric sensor disposed in, for example, the detection unit 120, each atherectomy device 10, or the drive device 100.

A change in shape of the shaft portion 20 is detected by the image sensor 126 configured to capture an image of the shaft portion 20 and disposed in, for example, the detection unit 120, each atherectomy device 10, or the drive device 100. The image sensor 126 is, for example, a camera.

The image sensor 126 may be used for detecting the size and quantity of a cut piece of an object. In this case, the image sensor 126 is disposed in a position where an image of the inside of the discharge channel 50 and the drainage bag 90 is captured from the outside.

Treatment target image information including a lesion is detected by the treatment target image sensor 128. The treatment target image sensor 128 detects two-dimensional or three-dimensional image information by, for example, intravascular ultrasound (IVUS), optical coherence tomography (OCT), image pickup by a camera, ultrasound diagnostic imaging, computed tomography (CT), or magnetic resonance imaging (MRI).

The treatment aid apparatus 110 includes the controller 111 with a memory and a processor. The memory stores programs and various parameters. The processor is, for example, a central processing unit (CPU) and configured to read programs or various parameters from the memory and perform arithmetic processing.

The treatment aid apparatus 110 also includes a display unit 112 such as a monitor to display information and an input unit 113 such as a touch panel, keyboard, and/or mouse, which enables an operator to operate and perform various settings.

The controller 111 includes an information management unit 114 that acquires, stores, and controls various information, a computing unit 115 that determines one or more appropriate atherectomy devices 10, and an information output unit 116 that causes the display unit 112 to display information about the determined atherectomy devices 10. The controller 111 performs at least part of the arithmetic processing with or without an external server to which the controller 111 is connected over a communication network.

The information received and controlled by the information management unit 114 includes device information A, treatment target information B, and treatment method information C. The device information A includes atherectomy device information, access device information, treatment device information, and treatment method information.

The atherectomy device information includes information associated with different types of atherectomy devices 10. The atherectomy device information includes the name and feature information of each type of atherectomy device 10. Herein, the names of devices and treatment methods are not particularly limited as long as the types of devices and treatment methods can be identified and may be, for example, product names, model numbers, abbreviations, or identifiers. The feature information indicates, for example, dimensions and properties of the atherectomy device 10. The dimensions are, for example, an effective length of each atherectomy device 10 in the longitudinal direction which is insertable into a blood vessel, presence or absence of a coating (such as diamond coating) applied to the cutting unit 30, the number of rotations of a motor, an outside diameter of the shaft portion 20, and an outside diameter of the cutting unit 30. The properties are, for example, a texture of a lesion to be cut (for example, a soft lesion such as plaque, a calcified hard lesion, and a mixed lesion of soft lesion and hard lesion), the site of a blood vessel to be treated, and an inside diameter of the blood vessel to be treated.

The access device information includes information associated with different types of access devices. The access device is a device capable of assisting each atherectomy device 10 to reach a desired position inside a body cavity, and examples of the access device include a guide wire, a guiding sheath, and an introducer kit. The access device information includes the name and feature information of each type of access device. The feature information indicates, for example, dimensions and properties of the access device. The dimensions are, for example, an effective length along the axis which is insertable into a blood vessel, an outside diameter, and an inside diameter. The properties are, for example, a blood vessel to be treated, and an inside diameter of the blood vessel to be treated.

The treatment device information includes information associated with different types of treatment devices. The treatment device is a device capable of performing treatment for a blood vessel having a lesion at least a part of which has been treated by an atherectomy device 10, and examples of the treatment device include a balloon catheter, a drug-coated balloon catheter having the balloon surface coated with a drug, a stent deployment catheter, and a cutting balloon catheter. The treatment device information includes the name and feature information of each type of treatment device. The feature information indicates, for example, dimensions and properties of the treatment device. The dimensions are, for example, an effective length of each treatment device in the longitudinal direction which is insertable into a blood vessel, an outside diameter of an expanded balloon or stent, an axial length of a balloon or stent, expansion pressure of a balloon, an outside diameter of a shaft, and an inside diameter of a guide wire lumen. The properties are, for example, the site of a blood vessel to be treated, an inside diameter of the blood vessel to be treated, presence or absence of a drug applied to the surface of a balloon or stent, the type of the drug, and a texture of a lesion to be cut (for example, a soft lesion, a hard lesion, and a mixed lesion).

The treatment method information C includes information associated with different types of treatment methods. The treatment method information includes the name and feature information of each type of treatment method. The feature information indicates, for example, a blood vessel to be treated, an inside diameter of the blood vessel to be treated, available atherectomy devices 10, available access devices, available treatment devices, a texture of a lesion (for example, a soft lesion such as plaque, a calcified hard lesion, and a mixed lesion of soft lesion and hard lesion).

The treatment target information B is, for example, information associated with an atherectomy device 10 previously used, information at the time of cutting by the previously used atherectomy device 10, information associated with a cut lesion, treatment target image information, information associated with the site of the lesion, evaluation information on the previously used atherectomy device 10 recorded by an operator, patient information, and other information.

The information at the time of cutting by the previously used atherectomy device 10 is detected by the detection unit 120 at the time of previous cutting and transmitted to the controller 111 and stored in the memory of the controller 111. The information at the time of the cutting indicates, for example, the number of rotations of the cutting unit 30, torque of the cutting unit 30, sounds and vibrations produced by the cutting unit 30 and the rotation drive unit 70, a value of current flowing into the rotation drive unit 70, an amount of energy supplied to the rotation drive unit 70, and a temperature around the rotation drive unit 70.

The information associated with the cut lesion is detected by the detection unit 120 such as the image sensor 126 and the treatment target image sensor 128 on completion of the cutting. The information associated with the cut lesion indicates, for example, a material of a cut piece of an object obtained by cutting the lesion, a shape of the cut piece, a quantity of the cut piece, and a patency rate. The shape and quantity of the cut piece are computed by known image recognition technology from the image information obtained by the image sensor 126. In a case where the cut piece is determined to have an elongated peel shape, the controller 111 determines that the lesion is a soft lesion or a mixed lesion. In a case where the cut piece is determined to have a particulate shape, the controller 111 determines that the lesion is a hard calcified lesion. The patency rate is computed by known image recognition technology from the treatment target image information obtained by the treatment target image sensor 128. The information associated with the site of the lesion is also computed by the known image recognition technology from the treatment target image information obtained by the treatment target image sensor 128.

The treatment target image information is, for example, cross-sectional image information obtained by ultrasound, and the controller 111 computes, for example, the hardness of the lesion obtained by ultrasound elastography. The hardness computed is typically represented by luminance in a two-dimensional image shown by the display unit 112.

The patient information indicates, for example, a patient's height, weight, blood pressure, medical history or anamnesis, and blood test result.

Other information includes, for example, a series of surgery information obtained from papers or academic information (such as patient information, sites and sizes of lesions, conditions of lesions, access devices, atherectomy devices, and treatment devices).

Furthermore, the controller 111 automatically performs cutting by controlling operations of the rotation drive unit 70, the aspiration drive unit 80, the axial direction position adjustment unit 101, and the rotation direction position adjustment unit 102.

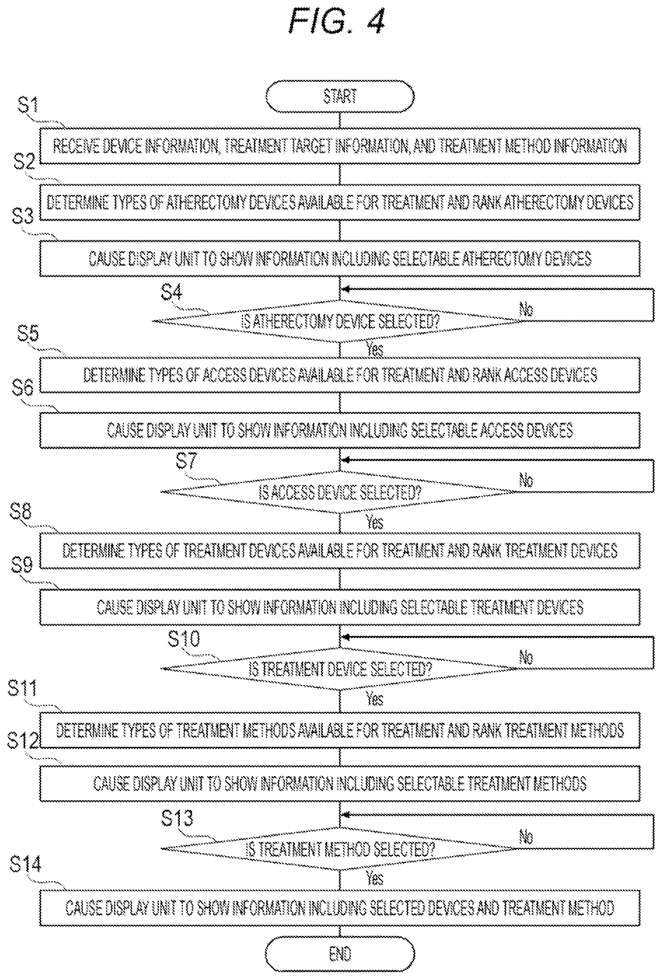

With reference to the flowchart in FIG. 4, the operation control of the treatment aid apparatus 110 will be described.

Before performing treatment with an atherectomy device 10, the information management unit 114 of the controller 111 acquires and stores the device information A, the treatment target information B, and the treatment method information C (S1).

Next, the computing unit 115 determines types of atherectomy devices 10 available for the treatment based on the atherectomy device information and the treatment target information B and computes a grade of recommendation for each atherectomy device 10, thereby ranking the atherectomy devices 10 (S2). The types of atherectomy devices 10 available for the treatment are determined by whether the devices satisfy use conditions included in the feature information of each atherectomy device 10 (such as the site of a blood vessel to be treated and an inside diameter of the blood vessel to be treated).

A method for computing a grade of recommendation is not particularly limited. For example, with regard to each item included in the feature information of each atherectomy device 10, the applicability to the treatment for a treatment target determined from the treatment target information B is analyzed and scored. The larger the total value of the items, the higher the grade of recommendation. For example, when the treatment target image information included in the treatment target information B is cross-sectional image information obtained by ultrasound, the controller 111 computes the hardness of a lesion obtained by ultrasound elastography. The higher the hardness of the lesion computed, the larger the controller 111 makes the number of rotations of the motor as the atherectomy device information, or the higher the controller 111 makes the grade of recommendation for a device having a diamond-coated cutting unit. With regard to the scoring of each item, for example, a desirable recommended inside diameter of a blood vessel is set within an inside diameter of a blood vessel to be treated included in feature information of a certain type atherectomy device 10. In a case where an inside diameter of a blood vessel determined by the treatment target information B falls within the recommended inside diameter, a predetermined high score is given, and conversely, the farther the inside diameter of the blood vessel is away from the recommended inside diameter, the lower the score. In this manner, it is possible to compute a grade of recommendation by setting a recommended range for each item and scoring the items. Note that the above algorithm for computing a grade of recommendation is described for illustration purposes and the algorithm is not limited to a specific one.

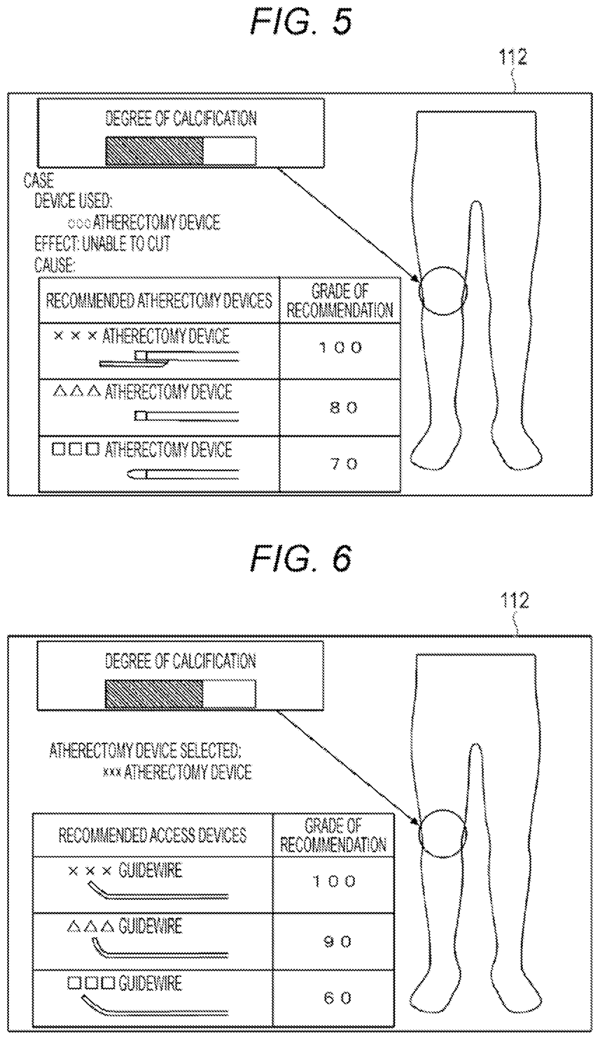

Next, as shown in FIG. 5, the information output unit 116 causes the display unit 112 to display a screen showing the names of different types of selectable atherectomy devices 10 together with their grades of recommendation (S3). The display unit 112 may show a photograph and a diagram of each atherectomy device 10. The display unit 112 may also show feature information of each atherectomy device 10. In addition, the display unit 112 may illustrate the site of the lesion and may also show a bar graph illustrating the degree of calcification of the lesion. The display unit 112 may also show the name indicating the type of an atherectomy device 10 previously used for cutting, an effect of the cutting, and a cause of the effect. The cause is an evaluation recorded by, for example, an operator who has done the cutting included in the treatment target information B.

The operator uses the input unit 113 to select one atherectomy device 10 through the screen displayed by the display unit 112. When one atherectomy device 10 is selected (S4), the controller 111 determines types of access devices available for the treatment based on the selected atherectomy device information, the access device information, and the treatment target information B and computes a grade of recommendation for each access device, thereby ranking the access devices (S5). Note that a grade of recommendation for an access device may be computed by an algorithm similar to one used for computing grades of recommendation for the atherectomy devices 10 or by other algorithms.

Next, as shown in FIG. 6, the information output unit 116 causes the display unit 112 to display a screen showing the name of the selected atherectomy device 10 together with the names of different types of selectable access devices and their grades of recommendation (S6). The display unit 112 may show a photograph and a diagram of each access device. The display unit 112 may also show feature information of each access device.

The operator uses the input unit 113 to select one access device through the screen displayed by the display unit 112. When one access device is selected (S7), the controller 111 determines types of treatment devices available for the treatment based on the selected access device information, the atherectomy device information, the treatment device information, and the treatment target information B and computes a grade of recommendation for each treatment device, thereby ranking the treatment devices (S8). Note that a grade of recommendation for a treatment device may be computed by an algorithm similar to one used for computing grades of recommendation for the atherectomy devices 10 or by other algorithms.

Next, as shown in FIG. 7, the information output unit 116 causes the display unit 112 to display a screen showing the names of the selected atherectomy device 10 and access device and show the names of different types of selectable treatment devices together with their grades of recommendation (S9). The display unit 112 may show a photograph and a diagram of each treatment device. The display unit 112 may also show feature information of each treatment device.

The operator uses the input unit 113 to select one treatment device through the screen displayed by the display unit 112. When one treatment device is selected (S10), the controller 111 determines types of treatment methods available for the treatment based on the selected treatment device information, the access device information, the atherectomy device information, the treatment target information B, and the treatment method information C and computes a grade of recommendation for each treatment method, thereby ranking the treatment methods (S11). Note that a grade of recommendation for a treatment method may be computed by an algorithm similar to one used for computing grades of recommendation for the atherectomy devices 10 or by other algorithms.

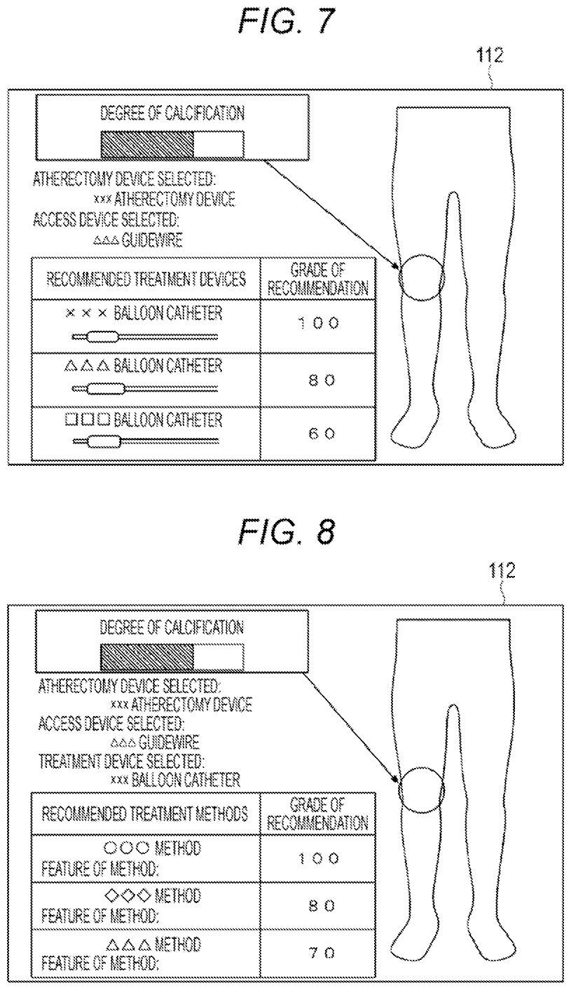

Next, as shown in FIG. 8, the information output unit 116 causes the display unit 112 to display a screen showing the names of the selected atherectomy device 10, access device, and treatment device together with the names of different types of selectable treatment methods and their grades of recommendation (S12). The display unit 112 may show feature information of each treatment method in a diagram or sentence. Alternatively, the information related to the treatment method shown on the display unit 112 does not necessarily include the name of the treatment method as long as the treatment method is determined.

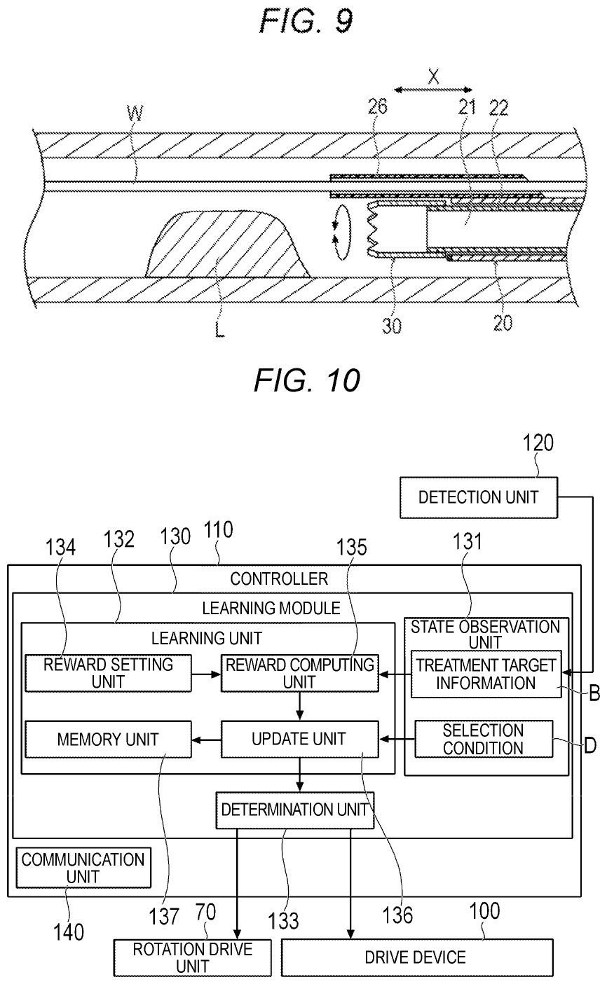

The operator uses the input unit 113 to select one treatment method through the screen displayed by the display unit 112. When one treatment method is selected (S13), the controller 111 causes the display unit 112 to show the selected atherectomy device 10, access device, treatment device, and treatment method (S14). Accordingly, the treatment aid apparatus 110 completes the operation control for selecting each device and a treatment method. After that, the operator cuts a lesion L by the selected atherectomy device 10, access device, treatment device, and treatment method. The controller 111 controls operations of the rotation drive unit 70, the aspiration drive unit 80, the axial direction position adjustment unit 101, and the rotation direction position adjustment unit 102 and causes the detection unit 120 to detect and store information while causing the cutting unit 30 to cut the lesion L as shown in FIG. 9.

As described above, the treatment aid apparatus 110 according to the first embodiment includes the controller 111 for aiding an operator in performing a treatment to cut a lesion L within a blood vessel of a patient. The controller 111 includes the information management unit 114 configured to acquire the device information A including the names and feature information of different types of devices and the treatment target information B including information associated with the lesion L, the computing unit 115 configured to rank the different types of devices appropriate for the treatment target information B based on the device information A and the treatment target information B, and the information output unit 116 configured to control the display unit 112 to display the ranked information.

The treatment aid apparatus 110 with the above configuration automatically determines types of appropriate devices depending on the patient's lesion L and causes the display unit 112 to show the information, so that it is possible to reduce burden on the operator.

The device information includes atherectomy device information including the names and feature information of different types of atherectomy devices 10, and the computing unit 115 ranks the different types of atherectomy devices 10 appropriate for the treatment target information B based on the atherectomy device information and the treatment target information B. Accordingly, the treatment aid apparatus 110 automatically determines types of appropriate atherectomy devices 10 depending on the patient's lesion L and causes the display unit 112 to show the information.

The treatment target information B includes information associated with the lesion L when being previously cut and/or the lesion L after being cut. Accordingly, in a case where cutting by a previously used atherectomy device 10 is inadequate, the effect of the previous cutting is considered and types of appropriate atherectomy devices 10 are displayed automatically.

The treatment target information B includes cross-sectional image information obtained by ultrasound, and the computing unit 115 computes the hardness of the lesion L based on the cross-sectional image information. The larger the hardness computed, the higher the computing unit 115 computes the rank of an atherectomy device having a motor with a larger number of rotations as the atherectomy device information or the rank of an atherectomy device having a diamond-coated cutting unit 30.

For example, the computing unit 115 acquires the luminance value (e.g., the greatest or average luminance value) of an image of the lesion L, and determines whether the luminance value exceeds a threshold (e.g., 128 when the luminance value is represented on a scale of 0 to 255). Upon determining that the luminance value exceeds the threshold, the computing unit 115 ranks the atherectomy device with a motor that can rotate at a speed greater than a predetermined speed (e.g., 20,000 rpm), higher than the atherectomy device with a motor that cannot rotate at that speed. Additionally or alternatively, upon determining that the luminance value exceeds the threshold, the computing unit 115 ranks the atherectomy device with a diamond-coated cutting unit 30 higher than the atherectomy device without a diamond-coated cutting unit 30. If there are two or more atherectomy devices with such a motor or cutting unit 30, the computing unit 115 ranks the atherectomy device with a motor that can rotate at a greater speed higher.

Accordingly, the treatment aid apparatus 110 automatically determines types of appropriate atherectomy devices 10 depending on the hardness of the patient's lesion L.

The device information includes access device information including the names and feature information of different types of access devices capable of assisting an atherectomy device 10 to reach a desired position, and the computing unit 115 ranks the many different types of access devices appropriate for the treatment target information B based on the access device information and the treatment target information B. Accordingly, the treatment aid apparatus 110 automatically determines types of access devices depending on the patient's lesion L and causes the display unit 112 to show the information.

The device information includes treatment device information including the names and feature information of different types of treatment devices available after cutting by an atherectomy device 10, and the computing unit 115 ranks the many different types of treatment devices appropriate for the treatment target information B based on the treatment device information and the treatment target information B. According, the treatment aid apparatus 110 automatically determines types of appropriate treatment devices depending on the patient's lesion L and shows the information on the display unit 112.

The computing unit 115 may compute a holding force for holding an atherectomy device 10 by the drive device 100 for driving the atherectomy device 10 that cuts the lesion L, and the information output unit 116 may control the display unit 112 to display information about the holding force. With this configuration, the display unit 112 shows the holding force appropriate for the selected atherectomy device 10. Accordingly, the treatment aid apparatus 110 automatically determines the holding force depending on the types of atherectomy devices 10 and causes the display unit 112 to show the information.

Furthermore, the treatment aid apparatus 110 according to this embodiment includes the controller 111 for aiding an operator in performing a treatment to cut a lesion L within a blood vessel of a patient. The controller 111 includes the information management unit 114 configured to acquire treatment method information including feature information of different types of treatment methods and the treatment target information B including information associated with the lesion L, the computing unit 115 that ranks the different types of treatment methods appropriate for the treatment target information B based on the treatment method information and the treatment target information B, and the information output unit 116 that controls the display unit 112 to display the ranked information.

The treatment aid apparatus 110 having the above configuration automatically determines types of appropriate treatment methods depending on the patient's lesion L and causes the display unit 112 to show the information, so that it is possible to reduce burden on an operator.

### Second Embodiment

A treatment aid apparatus 110 according to a second embodiment is different from the treatment aid apparatus 110 according to the first embodiment in that a controller 111 includes a learning module 130 that performs machine learning and a communication unit 140 that connects with a server for performing arithmetic processing in machine learning as shown in FIG. 10.

Machine learning algorithms are typically classified into supervised learning, unsupervised learning, and reinforcement learning. The supervised learning algorithm is used to perform machine learning based on a set of input data and result data given. The unsupervised learning algorithm is used to perform machine learning based on a large amount of input data given. The reinforcement learning algorithm is used to change the environment, which corresponds to the treatment target information B described above, based on a solution, which corresponds to types of available devices and treatment methods, and grades of recommendation for each device and each treatment method described above, output by the algorithm, thereby correcting the solution based on a reward, that is, the accuracy of the output solution. In this embodiment, reinforcement learning is used as the machine learning. Note that machine learning other than reinforcement learning may be used in the invention.

The learning module 130 includes a state observation unit 131, a learning unit 132, and a determination unit 133. The learning module 130 corresponds to an agent in reinforcement learning. The learning module 130 is performed by the controller 110 or an external device connectable to, for example, the treatment aid apparatus 111 according to the first embodiment.

The state observation unit 131 detects environmental states. The state observation unit 131 observes state variables including the treatment target information B and selection conditions D (e.g., types of available devices and treatment methods, and grades of recommendation for each device and each treatment method). The state observation unit 131 correlates the treatment target information B with the selection conditions D and stores the information.

The learning unit 132 includes a reward setting unit 134 that sets reward conditions, a reward computing unit 135 that computes a reward, an update unit 136 that updates a function, and a memory unit 137 that stores an outcome of learning by the update unit 136.

The reward setting unit 134 sets reward conditions. The reward conditions set by the reward setting unit 134 are determined by, for example, the stability of the treatment target information B, the time required for each procedure or treatment, the quality of each procedure or treatment, the energy expenditure, and the correspondence between the grades of recommendation for each device and each treatment method and the types of devices and treatment method that are actually selected by an operator. For example, in a case where the treatment target information B is determined to be stable (i.e., the fluctuation of the treatment target information B is determined to be small) the reward increases, and in the reverse case the reward decreases. In addition, in a case where the time required for treatment is long the reward decreases, and in the reverse case the reward increases. Furthermore, in a case where the quality of treatment enhances, the reward increases, and in the reverse case the reward decreases. Still further, the reward increases in a case where the grades of recommendation for the types of devices and treatment method that are actually selected by an operator are high, and in the reverse case the reward decreases. To determine in this manner, there are provided units for acquiring each data, and each data has an individual threshold set in advance. The time required for the treatment is determined by the controller 111, and the energy expenditure is determined by a current sensor 123 and a voltage sensor 124. The quality of treatment is computed by, for example, an image obtained by an image sensor 126. The correspondence between the grades of recommendation for each device and each treatment method and the types of devices and treatment method that are actually selected by an operator is determined by the controller 111.

The reward computing unit 135 computes a reward based on at least one treatment target information B and reward conditions observed by the state observation unit 131.

The update unit 136 updates a function for determining the selection condition D from the current state variable based on the reward computed by the reward computing unit 135. The function is, for example, an action value function.

The determination unit 133 learns selection of a high-value action. Based on an outcome of learning by the learning unit 132, the determination unit 133 determines at least one selection condition D and an optimal amount of correction of this selection condition D from the current state variable.

The communication unit 140 is connectable to a server over a network. Accordingly, the controller 111 performs at least part of high-load arithmetic processing in machine learning using the server over a network. Therefore, the controller 111 of the medical system 1 or the treatment aid apparatus 110 connectable to the server performs arithmetic processing for computing an optimal action value function and a state variable using the server over a network and receives a result of the arithmetic processing over the network, thereby updating the result optimally.

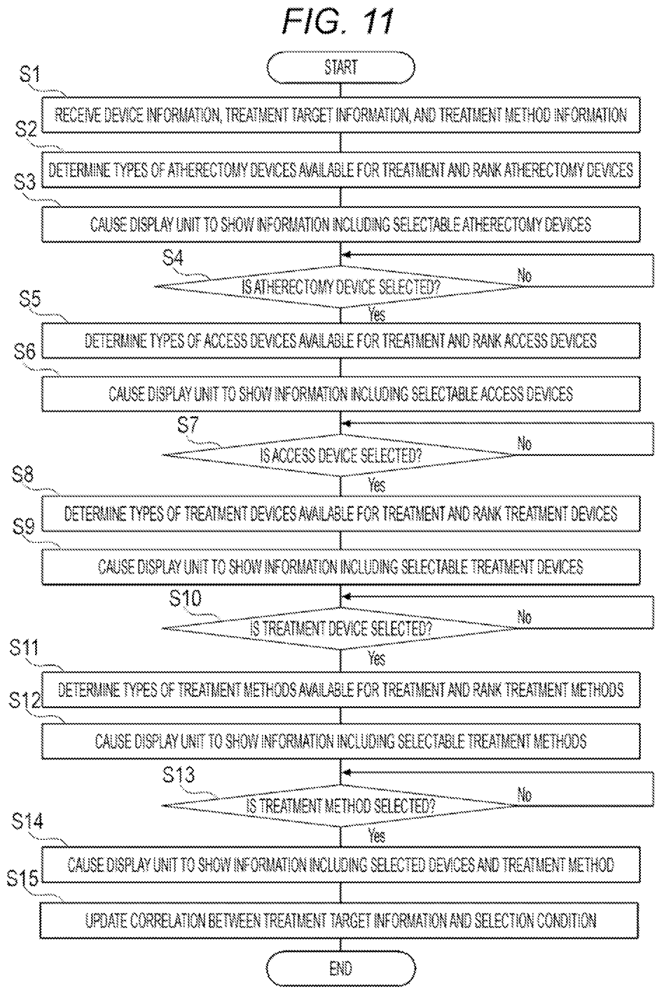

As shown in FIG. 11, the processing of the learning module 130 is performed every time the treatment aid apparatus 110 treats a lesion L (S15). The reward computing unit 135 computes a reward value in each selection condition D and the update unit 136 updates the action value function to increase the reward value. The action value function is updated by, for example, a known update formula used in Q learning. Accordingly, the learning module 130 updates at least one selection condition D to cut the lesion L.

Repeatedly performing machine learning for each cutting operation enhances the reliability of the action value function. Using the highly reliable action value function further optimizes at least one selection condition D to increase, for example, a Q-value used in Q-learning. Accordingly, when performing the cutting, the highly reliable action value function is used to automatically produce an optimal selection condition D from a detected physical quantity A, whereby the display unit 112 shows the information in a selectable manner. Note that the update unit 136 may update the action value function not for each cutting operation but during each operation.

In the second embodiment, the controller 111 includes the learning unit 132 that learns to determine at least one selection condition D by updating a function that determines at least one selection condition D based on a state variable including at least one treatment target information B and at least one selection condition D. The learning unit 132 includes the reward computing unit 135 that computes a reward for a result used to determine at least one selection condition D based on the state variable and the update unit 136 that updates a function based on the reward computed by the reward computing unit 135. The learning unit 132 repeatedly updates the function by the update unit 136 to learn at least one selection condition D that maximizes the reward. For this reason, the medical system 1 repeatedly perform machine learning to enhance the reliability of the function used to determine the selection condition D. Based on the highly reliable function for determining the selection condition D, at least one selection condition D is optimized.

The invention is not limited to the above embodiments, and various modifications may be made within the technical idea of the invention by those skilled in the art. For example, the cutting unit 30 is not necessarily a rotative body and may be, for example, a cutting structure that moves along the axial direction X or a member that emits a laser beam. In the above embodiments, the atherectomy devices 10, the access devices, the treatment devices, and the treatment methods are all ranked but at least one of them may be ranked. Alternatively, the order of ranking may be different.

What is claimed is:

1. A medical system for removing an object in a body cavity, comprising:
a display; and
a controller connectable to a drive device configured to control a medical device operated by an operator to remove the object, wherein
the controller is configured to:
acquire device information indicating features of different types of first devices connectable to the medical device,
acquire target information indicating characteristics of a target object to be removed,
rank the first devices for removing the target object using the device information and the target information, and
display on the display a list of the first devices according to the rank thereof.

2. The medical system according to claim 1, wherein the first devices include atherectomy devices each having a rotatable drive shaft, a motor configured to rotate the drive shaft, and a cutter attached to a distal end of the drive shaft and configured to cut the object when the drive shaft is rotated by the motor.

3. The medical system according to claim 2, wherein
the target information includes a cross-sectional image of the target object, and
the controller is configured to:
determine hardness of the target object based on the cross-sectional image, and
when the hardness exceeds a threshold, rank an atherectomy device having a motor that can rotate at a speed greater than a predetermined speed, higher than an atherectomy device having a motor that cannot rotate at a speed greater than the predetermined speed.

4. The medical system according to claim 3, wherein the cross-sectional image is an ultrasound image.

5. The medical system according to claim 2, wherein
the target information includes a cross-sectional image of the target object, and
the controller is configured to:
determine hardness of the target object based on the cross-sectional image, and
when the hardness exceeds a threshold, rank an atherectomy device having a diamond-coated cutter higher than an atherectomy device not having a diamond-coated cutter.

6. The medical system according to claim 2, wherein
the device information indicating features of different types of second devices by which an atherectomy device is guided toward the target object, and
the controller is configured to:
rank the second devices using the device information and the target information, and
display on the display a list of the second devices according to the rank thereof.

7. The medical system according to claim 6, wherein
the device information indicating features of different types of third devices for treating the body cavity from which the target object has been removed by an atherectomy device, and
the controller is configured to:
rank the third devices using the device information and the target information, and
display on the display a list of the third devices according to the rank thereof.

8. The medical system according to claim 7, wherein the controller displays on the display the lists of the first, second, and third devices on different screens in this order.

9. The medical system according to claim 1, wherein
the drive device is configured to hold and control a first device connected to the medical device, wherein
the controller is configured to:
detect a selection of one of the first devices listed on the display,
determine a holding force for holding the selected first device by the drive device, and
display information about the holding force on the display.

10. The medical system according to claim 1, wherein the target information further indicates characteristics of the target object before a treatment that has been previously performed.

11. A treatment aid apparatus for aiding an operator of a medical device to remove an object in a body cavity, comprising:
a display; and
a processor configured to:
acquire device information indicating features of different types of first devices connectable to the medical device,
acquire target information indicating characteristics of a target object to be removed,
rank the first devices for removing the target object using the device information and the target information, and
control the display to display a list of the first devices according to the rank thereof.

12. The treatment aid apparatus according to claim 11, wherein the first devices include atherectomy devices each having a rotatable drive shaft, a motor configured to rotate the drive shaft, and a cutter attached to a distal end of the drive shaft and configured to cut the object when the drive shaft is rotated by the motor.

13. The treatment aid apparatus according to claim 12, wherein
the target information includes a cross-sectional image of the target object, and
the processor is configured to:
determine hardness of the target object based on the cross-sectional image, and
when the hardness exceeds a threshold, rank an atherectomy device having a motor that can rotate at a speed greater than a predetermined speed, higher than an atherectomy device having a motor that cannot rotate at a speed greater than the predetermined speed.

14. The treatment aid apparatus according to claim 13, wherein the cross-sectional image is an ultrasound image.

15. The treatment aid apparatus according to claim 12, wherein
the target information includes a cross-sectional image of the target object, and
the processor is configured to:
determine hardness of the target object based on the cross-sectional image, and
when the hardness exceeds a threshold, rank an atherectomy device having a diamond-coated cutter higher than an atherectomy device not having a diamond-coated cutter.

16. The treatment aid apparatus according to claim 12, wherein
the device information indicating features of different types of second devices by which an atherectomy device is guided toward the target object, and
the processor is configured to:
rank the second devices using the device information and the target information, and
control the display to display a list of the second devices according to the rank thereof.

17. The treatment aid apparatus according to claim 16, wherein
the device information indicating features of different types of third devices for treating the body cavity from which the target object has been removed by an atherectomy device, and
the processor is configured to:
rank the third devices using the device information and the target information, and
control the display to display a list of the third devices according to the rank thereof.

18. The treatment aid apparatus according to claim 17, wherein the processor controls the display to display the lists of the first, second, and third devices on different screens in this order.

19. The treatment aid apparatus according to claim 11, wherein
the treatment aid apparatus is connectable to a drive device configured to hold and control a first device connected to the medical device, and
the processor is configured to:
detect a selection of one of the first devices listed on the display,
determine a holding force for holding the selected first device by the drive device, and
control the display to display information about the holding force.

20. A treatment aid apparatus for aiding an operator of a medical device to remove an object in a body cavity, comprising:
a display; and
a processor configured to:
acquire treatment method information indicating features of different types of methods for removing an object in a body cavity,
acquire target information indicating characteristics of a target object to be removed,
rank the treatment methods for removing the target object using the treatment method information and the target information, and
control the display to display a list of the methods according to the rank thereof.
